(19) United States
(12) Patent Application Publication
(10) Pub. No.: US 2026/0108615 A1
(43) Pub. Date: Apr. 23, 2026

(54) SELF-ASSEMBLED MUCOADHESIVE BIOPOLYMER PARTICLE RELEASE SYSTEM AND PREPARATION METHOD THEREOF

(71) Applicant: SZEGEDI TUDOMÁNYEGYETEM, Szeged (HU)

(72) Inventors: Mohamed M ABDELGHAFOUR, Szeged (HU); Diána SZABÓ, Szeged (HU); László JANOVÁK, Szeged (HU); Imre DÉKÁNY, Szeged (HU); László ROVÓ, Szeged (HU)

(21) Appl. No.: 18/697,611

(22) PCT Filed: Sep. 29, 2022
(86) PCT No.: PCT/HU2022/050069

§ 371 Date: Apr. 1, 2024

(30) Foreign Application Priority Data
Oct. 1, 2021 (HU) P2100345

(51) Int. Cl. A61K 47/58; A61K 9/00; A61K 9/51; A61K 31/05; A61K 31/145; A61K 31/192; A61K 31/704; A61K 47/69; B82Y 5/00; B82Y 40/00

(52) U.S. Cl. CPC A61K 47/58; A61K 9/006; A61K 9/5192; A61K 31/05; A61K 31/145; A61K 31/192; A61K 31/704; A61K 47/6937; B82Y 5/00; B82Y 40/00

(57) ABSTRACT

The present invention relates to a mucoadhesive polymeric prodrug comprising a partially succinated polyvinyl alcohol (PVA-SA) with adjusted amount of hydroxyl and carboxyl pendant groups, which form an ester and/or amide linkage with amino and/or carboxyl and/or hydroxyl groups of biologically active compounds such as proteins, peptides, synthetic chemical, or natural products compounds (e.g. doxorubicin as an antitumor agent and antifibrotic drug). Preferably, said biologically active compounds are cysteamine (CYS) as the aminothiol compound and one compound selected from the group consisting of doxorubicin (DOX), ketoprofen (KETO), or 4-hydroxybenzyl alcohol (HBA). Moreover, the simultaneous presence of both hydroxyl and carboxyl groups in the partially succinated polyvinyl alcohol (PVA-SA) chain of the prodrug enables the self-assembled formation of 50-260 nm particles from the linear macromolecules and thus the drug release can be prolonged or adjusted. The present invention also relates to improving the mucoadhesive properties of the polymeric prodrug by the regulation of the amount of conjugated aminothiol compound. Further, the present invention relates to the method for producing said mucoadhesive polymeric prodrug, and nanoparticles of the said mucoadhesive polymeric prodrug.

### FIELD OF THE INVENTION

The present invention relates to a mucoadhesive polymeric prodrug comprising a partially succinated polyvinyl alcohol (PVA-SA) with adjusted amount of hydroxyl and carboxyl pendant groups, which form an ester and/or amide linkage with amino and/or carboxyl and/or hydroxyl groups of biologically active compounds such as proteins, peptides, synthetic chemical, or natural products compounds (e.g. doxorubicin as an antitumor agent and antifibrotic drug). More particularly, the present invention relates to self-assembled nanoparticles of mucoadhesive polymeric prodrug comprising a partially succinated polyvinyl alcohol (PVA-SA) and biologically active compounds having at least one group selected from amino, carboxyl, and hydroxyl groups, where the biologically active compounds is linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds, and wherein one of the biologically active compounds is an aminothiol compound, and wherein the polymeric prodrug has crosslinking ester groups formed between the carboxyl and hydroxyl groups of the partially succinated polyvinyl alcohol. Preferably, said biologically active compounds are cysteamine (CYS) as the aminothiol compound and one compound selected from the group consisting of doxorubicin (DOX), ketoprofen (KETO), or 4-hydroxybenzyl alcohol (HBA). Moreover, the simultaneous presence of both hydroxyl and carboxyl groups in the partially succinated polyvinyl alcohol (PVA-SA) chain of the prodrug enables the self-assembled formation of 50-260 nm particles from the linear macromolecules and thus the drug release can be prolonged or adjusted. The present invention also relates to improving the mucoadhesive properties of the polymeric prodrug by the regulation of the amount of conjugated aminothiol compound. Further, the present invention relates to the method for producing said mucoadhesive polymeric prodrug, and nanoparticles of the said mucoadhesive polymeric prodrug.

### BACKGROUND OF THE INVENTION

Doxorubicin (DOX) is an anthracycline antibiotic, is regarded to be one of the most potent chemotherapeutic drugs approved by the Food and Drug Administration (FDA), which was first extracted from Streptomyces peucetius var. caesius early in the 1960s and has been used as an effective treatment against several cancers [H. G. Keizer et al., Pharmacol. Ther. 1990, 47, 219-231; K. Renu et al., Eur. J. Pharmacol. 2018, 818, 241-253]. When administered as the main treatment, it has demonstrated promising results in adult and childhood cancers, including both solid tumors and hematological malignancies [H. G. Keizer et al., Pharmacol. Ther. 1990, 47, 219-231; G. Minotti et al., Pharmacol. Rev. 2004, 56, 185-229; A. M. Meredith et al., J. Pharm. Pharmacol. 2016, 68, 729-741]. It is usually used to treat breast cancers [H. Sonowal et al., Sci. Rep. 2017, 7, 3182; Y. Shi et al., Int. J. Clin. Exp. Pathol. 2018, 11, 2347-2355], multiple myelomas [H. Cortes-Funes and C. Coronado, Cardiovasc. Toxicol. 2007, 7, 56-60], soft tissue sarcomas [H. Cortes-Funes and C. Coronado, Cardiovasc. Toxicol. 2007, 7, 56-60; Carvalho C et al., Curr. Med. Chem. 2009, 16, 3267-3285], non-Hodgkin lymphomas [B. Guo et al., Onkologie. 2011, 34, 184-188], childhood solid tumors [N. M. Marina et al., Clin. Cancer Res. 2002, 8, 413-418], lung cancers [C. F. Thorn et al., Pharmacogenet. Genomics 2011, 21, 440-446] and acute leukemia's [A. Ruggiero et al., Int. J. Clin. Oncol. 2013, 18, 927-933]. Despite the fact that DOX has shown tremendous efficacy in killing rapidly dividing cells and delaying the progression of solid and liquid tumors, drug resistance and various side effects develop during the DOX treatment, making it a significant limitation as an effective cancer treatment [C. F. Thorn et al., Pharmacogenet. Genomics 2011, 21, 440-446; F. Yang. et al., Biochim. Biophys. Acta 2014, 1845, 84-89; A. Varela-Lopez et al., Food Chem. Toxicol. 2019, 134, 110834]. DOX-induced cardiotoxicity is a significant issue that needs the development of novel forms to reduce the risk of morbidity. Therefore, it is necessary to develop prodrugs or so-called drug delivery systems (DDS) to provide the prolonged therapeutic effect of DOX and safe administration to reduce its side effect and enhance therapeutic efficiency.

A polymeric prodrug is a conjugation of a drug with a polymer. A prodrug is a type of drug which remains inactive when administered to the site of action and is activated on the targeted site by specific conditions. Based on the previous definition, a prodrug is considered an inactive precursor of a drug. Albert and Harper first introduced the concept of the prodrug [A. Albert, Nature 1958, 182, 421-423; N. J. Harper, J. Med. Chem. 1958, 1, 467-500; K. Hoste et al., Int. J. Pharm. 2004, 277, 119-131]. Polymeric prodrugs have several advantages include prolongation of drug action, controlled drug release, and immuno-protection during cancer treatment. According to Ringsdorfs model [H. Ringsdorf, J. Polym. Sd., Polym. Symp. 1975, 51, 135-153], polymeric prodrug mainly contains five components: polymeric backbone, spacer, drug, soluble agent, and targeting group. A coupling reaction includes the conjugation of a drug with a polymer. 1-Ethyl-3-(3-dimethyl aminopropyl) carbodiimide (EDC, EDCl), dicyclohexylcarbodiimide (DCC), and N-hydroxy succinimide esters are among the most widely used couple agents. Drugs or any other active agents are chemically conjugated via disulfide, amide, or ester bonds to polymers. Until reaching the desired site, the bonding must be comparatively stable to avoid drug release during its transport. The coupling of drugs with polymer via an amide bond is strongly promising due to its high stability against different reaction conditions and high temperature [S. Mahesh et al., Molecules 2018, 23, 2615-2657]. In this scenario, there is a chance for the administration of a comparatively significant amount of prodrug without concomitant side effects and overdosing risk. The drug is released over time which thereby reduces the need to repeatedly administration the drug.

Mucoadhesion is the natural or synthetic polymer attachment phenomenon to a mucosal surface. The mucosal layer is presented in different body regions, such as the gastrointestinal tract, airways, urogenital tract, ear, eye, and nose. Thus, mucoadhesive drug delivery systems (MDDS) may be configured to specifically target oral, rectal, buccal, vaginal, ocular, and nasal routes of administration. MDDS provides several advantages over other conventional delivery systems, for example increasing drug residence at the targeted site, increasing drug permeability, and increasing drug bioavailability [I. Singh and V. Rana, J. Adhes. Sci. Technol. 2012, 26, 2251-2267]. Thiolate polymers are a kind of mucoadhesive polymers of the second generation which are derived from hydrophilic polymers including chitosan polyacrylate or gellan gum deacetylates [E. Mathiowitz et al., Bioadhesive drug delivery systems. In: Mathiowitz E, editors. Encyclopedia of Controlled Drug Delivery, vol. 1. New York: Wiley; 1999. p. 9-44]. Due to the existence of thiol groups, thiolate polymers can be easy form covalent bonds (disulfide bonds) with cysteine-rich sub-domains of the mucus gel layer, in addition to the hydrogen-bonds and van der Waals' forces which lead to increasing the drug residence time and enhance bioavailability [K. Albrecht et al., J. Control. Release 2006, 115, 78-84]. Cysteamine (CYS) or 2-mercapto ethylamine is an endogenous aminothiol compound synthesized during the co-enzyme A metabolism cycle by human body cells. CYS was chosen as a source of thiol group and improvement of mucoadhesive properties of polymeric prodrug due to ease attached to the polymer with the carboxylic group by the formation of an amide bond by its amino group and the carboxylic group. Unlike cysteine, cysteamine does not have a carboxylic acid group, that can exclude undesired side reactions, including CYS-CYS amide bonds formation or an ester bond formation with the hydroxyl group of the applied polymer [A. Bernkop-SchnGrch and S. Steininger, Int. J. Pharm. 2000, 194, 239-247].

Polyvinyl alcohol (PVA) is one of the water-soluble synthetic polymers most widely used in biomedical applications. PVA provides a suitable base for drug delivery systems (DDS) due to its good mechanical properties, biodegradability, biocompatibility, and ease of modification [E. Chiellini et al., Prog. Polym. Sd. 2003, 28, 963-1014; S. Moulay, Polym. Plast. Technol. Eng. 2015, 54, 1289-1319]. Biodegradable properties of the polymer are very important for drug delivery applications due to detecting the disposal process of the polymer after the delivery of the drug to the desired site and the possibility of utilizing it. Many studies of PVA degradation have been significantly increased [Y. Kawagoshi and M. Fujita, World J. Microbiol. Biotechnol. 1998, 14, 95-100; S. Matsumura et al., Macromolecules 1999, 32, 7753-7761; Y. Zhang et al., World J. Microbiol. Biotechnol. 2006, 22, 625-628; J. Chen et al., Enzyme Microb. Technol. 2007, 40, 1686-1691; B. Tang et al., Polym. Degrad. Stab. 2010, 95, 557-563; M. Li et al., J. Microbiol. Biotechnol., 2012, 22, 220-225] after the discovery of the first PVA-degrading bacteria by T. Suzuki et al. [Agric. Biol. Chem., 1973, 37, 747-756]. The common metabolic pathway of PVA by different organisms mainly includes two degradation steps; oxidation of hydroxyl groups by secondary alcohol oxidase (SAO) or PVA dehydrogenase (PVADH) by depending on pyrroloquinoline quinone (PQQ) in case of periplasmic degradation and hydrolytic cleavage of a C—C bond at diketone structures by s-diketone hydrolase (BDH) also called OPH [N. Ben Halima, RSC Adv. 2016, 6, 39823-39832]. PVA has FDA approval for in vivo applications and clinical uses in the human body [M. I. Baker et al., J. Biomed. Mater. Res. B Appl. Biomater. 2012, 100, 1451-1457; S.-F. Chong et al., Small 2013, 9, 942-950]. The biocompatibility of PVA has been demonstrated in 1975 by Tadavarthy et al. [Am. J. Roentgenol. 1975, 125, 609-616] in addition to preclinical and clinical studies of biocompatibility of PVA confirmed the safety and biocompatibility without any adverse side effects in the surrounding tissue, evidence of tissue loss and cytotoxicity as was reported by Baker et al. [J. Biomed. Mater. Res. B Appl. Biomater. 2012, 100, 1451-1457]. Due to the features of the above PVA, mucoadhesive/bioadhesive properties of PVA can be used during the drug release process. In addition to its biomedical applications (e.g. wound dressings, contact lenses, drug delivery systems), PVA plays an important role in cosmetics (such as body and skincare) [M. A. Nilforoushzadeh et al., J. Cosmet. Dermatol. 2018, 17, 693-702], electronics [B. Chaudhuri et al., Colloids Surf B 2016, 143, 71-80] and is also widely used in tissue engineering applications, including the fabrication of vascular tissue, artificial cartilage, bone tissue, etc [T. Noguchi et al., J. Appl. Biomater. 1991, 2, 101-107; L. V. Thomas et al., J. Mater. Sd. Mater. Med. 2009, 20, 259-269; G. Leone et al., J. Biomed. Mater. Res.—Part B Appl. Biomater. 2011, 97, 278-288; S. Chahal et al., Procedia Eng. 2013, 53, 683-688; E. Yan et al., Mater. Sd. Eng. C. 2014, 41, 217-223; E. A. Kamoun et al., Arab. J. Chem. 2015, 8, 38-47; P. Picone et al., Int. J. Biol. Macromol. 2019, 121, 784-795].

Polymers that contain OH groups (e.g. PVA) can be readily converted by treatment with acid anhydrides into carboxylic acid derivatives [C. L. Crane and D. F. ingerick, Manufacture of Dicarboxylic Acid Esters of Polyvinyl Alcohols (GB1181000A patent application), issued Feb. 11, 1970; J. Ikeda and K. Kuragaki, Partial Esterification of Polyvinyl Alcohol JP2000239317A patent application), issued Sep. 5, 2002; R. Jantas et al., Am. J. Polym. Sd. 2012, 2, 79-84; S. A. Castleberry et al., Nano-Fibular Nanoparticle polymer-drug conjugate for sustained Dermal Delivery of Retinoids (US2018185513A1 patent application), issued Jul. 5, 2018; N. MURTHY et al., AntiMicrobial Agent-Polymer Conjugates and Methods of Use Thereof (WO2018195078A-1 patent application), issued Oct. 25, 2018: K. S. Lam et al, Poly(Vinyl Alcohol) Nanocarriers (WO2017031084A1 patent application), issued Feb. 23, 2017]. Anhydrides (e.g. succinic anhydride, SA) may be used to form a PVA ester that terminates with free carboxylate groups [K. M Camacho et al., Nanomedicine 2016, 11, 1139-1151]. For drug and protein conjugation, the resulted succinated derivative with the free (COOH) group is further used [K. M Camacho et al., Nanomedicine 2016, 11, 1139-1151; P. Jayaraman et al., Bull. Mater. Sd. 2016, 39, 201-207; G. Rivera-Hernindez et al., Int. J. Pharm. 2021, 600, 120478]. However, these preparation procedures provide linear macromolecules with conjugated drugs or other active agents. The succinyl group integrated into the polymer can work as a spacer between both the drug and the polymer, which controls the rate release through enzymatic and/or hydrolytic (non-enzymatic) cleavage of the temporary bond between the drug and polymer. Under the in vitro environment (enzyme-free condition) via using aqueous buffer solution, the rate of hydrolysis of the temporary bond (e.g. amide or ester bond) may be too slow and not therapeutically effective. However, the presence of amidase or esterase in an in vivo environment will result in a significant catalytic acceleration of hydrolysis kinetics from twofold to multiple magnitude orders [R. B. Greenwald et al., J. Med. Chem. 1999, 42, 3857-3867].

### The Problem to be Solved by the Inventions

The object of the present invention is to provide self-assembled mucoadhesive nanoparticles of mucoadhesive polymeric prodrug and its production method. Said nanoparticles of mucoadhesive polymeric prodrug should have the following features:
a) they are in the form of nanosized particles that enhance the surface area and thus the availability of drug and prevents accumulation or aggregation;
b) they deliver slow-releasing of biologically active compound from the polymeric prodrug that enhances the prolongation of therapeutic effect and avoids frequently repeated-dose administration;
c) they have mucoadhesive properties that enhance the residence time and avoid rapid elimination; and
d) they are inactive form (prodrug) suitable for any type of administration routes to avoid the damage of tissue during the transportation and the hydrophobization that enhanced permeability and retention (EPR) effect.

### The Discovery According to the Present Invention

To achieve the above-mentioned objective, we have carried out systematic experimental work, which has resulted in our invention. The present invention is based on the unexpected discovery that if a polymeric prodrug is prepared by the method of the invention, the conjugation reaction of biologically active compounds having at least one group selected from amino, carboxyl, and hydroxyl groups (e.g. doxorubicin (DOX) having an amino group) able to form an amide or ester linkage with the partially succinated PVA, will be accompanied by the self-assembling formation of nanoparticles by crosslinking ester groups formed between the carboxyl and hydroxyl groups of the partially succinated polyvinyl alcohol.

### BRIEF DESCRIPTION OF THE INVENTION

1. A mucoadhesive polymeric prodrug comprising
a) a partially succinated polyvinyl alcohol (PVA-SA) polymer;
b) two or more biologically active compounds having at least one functional group selected from amino, carboxyl, and hydroxyl groups;
wherein one of the biologically active compounds is an aminothiol compound, and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
and wherein the polymeric prodrug has crosslinking ester groups formed between the carboxyl and hydroxyl groups within the structure of the partially succinated polyvinyl alcohol.

2. The mucoadhesive polymeric prodrug according to point 1, wherein the molecular weight of the partially succinated PVA-SA polymer is between 20-80 kDa, more preferably 40-60 kDa.

3. The mucoadhesive polymeric prodrug according to point 1 or 2, wherein the substitution (succination) degree in the partially succinated PVA-SA polymer is below 10 mol %.

4. The mucoadhesive polymeric prodrug according to points 1 to 3, wherein the cross-link density is 1-9% in the partially succinated PVA-SA polymer.

5. The mucoadhesive polymeric prodrug according to points 1 to 4, wherein the biologically active compounds are cysteamine (CYS) as the aminothiol compound and one compound selected from the group consisting of doxorubicin (DOX), ketoprofen (KETO) and 4-hydroxybenzyl alcohol (HBA), more preferably DOX.

6. The mucoadhesive polymeric prodrug according to points 1 to 5, wherein DOX and cysteamine (CYS) constitute approx. 3.3% and 6.2% by weight of the formulation, respectively, referred to the total amount of the polymeric prodrug, when the carboxylic content is approx. 9% by weight.

7. Self-assembled nanoparticles of mucoadhesive polymeric prodrug comprising
a) a partially succinated PVA-SA polymer;
b) two or more biologically active compounds having at least one functional group selected from amino, carboxyl, and hydroxyl groups;
wherein one of the biologically active compounds is an aminothiol compound,
and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
and wherein the polymeric prodrug has crosslinking ester groups formed between the carboxyl and hydroxyl groups within the structure of the partially succinated polyvinyl alcohol.

8. The self-assembled nanoparticles of mucoadhesive polymeric prodrug according to point 7, wherein the size of the nanoparticles of the mucoadhesive polymeric prodrug is at most 260 nm, preferably 50 nm to 260 nm, the substitution degree is below 10% and the partially succinated PVA concentration is lower than 3% by weight.

9. The self-assembled nanoparticles of mucoadhesive polymeric prodrug according to point 7 or 8, wherein the nanoparticles have a size of 50-260 nm.

10. The self-assembled nanoparticles of mucoadhesive polymeric prodrug according to points 7 to 9, wherein the molecular weight of the partially succinated PVA-SA polymer is approx. 46 kDa.

11. The self-assembled nanoparticles of mucoadhesive polymeric prodrug according to point 7 to 10, wherein the substitution (succination) degree in the partially succinated PVA-SA polymer is below 10 mol %.

12. The self-assembled nanoparticles of mucoadhesive polymeric prodrug according to points 7 to 11, wherein the cross-link density is 1-9% in the partially succinated PVA-SA polymer.

13. The self-assembled nanoparticles of mucoadhesive polymeric prodrug according to points 7 to 12, wherein the biologically active compounds are CYS as the aminothiol compound and one compound selected from the group consisting of DOX, KETO and HBA, more preferably DOX.

14. The self-assembled nanoparticles of mucoadhesive polymeric prodrug according to points 7 to 13, wherein DOX and CYS constitute approx. 3.3% and 6.2% by weight of the formulation, respectively, referred to the total amount of the polymeric prodrug, when the carboxylic content is approx. 9% by weight.

15. A process for producing the mucoadhesive polymeric prodrug according to points 1 to 6, or the self-assembled nanoparticles of mucoadhesive polymeric prodrug according to points 7 to 14, wherein the following steps are taken:
i) dispersing 10-20 μm PVA particles in an organic solvent and then adding calculated amount of succinic anhydride between 1 to 60 molar %, preferably 3 to 55 molar %, in particular e.g. 3.4 to 53.8 molar % to hydroxyl content, and a catalyst;
ii) optionally stirring the reaction mixture obtained in step i);
iii) optionally removing the unreacted compounds and quenching the reaction of the solution obtained in step ii) and decanting the solvent to obtain particles of the partially succinated PVA;
iv) optionally purifying the polymer obtained in step iii) and drying under the vacuum;
v) optionally dissolving the modified polymer obtained in step iv) and all of the biologically active compounds used in distilled water;
vi) adding a coupling agent, preferably in approx. 50 mol % excess referred to COOH content, optionally with a catalyst to the solution obtained in step v);
vii) optionally stirring the solution obtained in step vi);
viii) optionally removing the unreacted biologically active compounds and the coupling agent from the solution obtained in step vii) to obtain the desired polymeric prodrug;
ix) optionally purifying the obtained polymeric prodrug obtained in step viii) and drying under the vacuum.

16. The process as claimed in point 15, wherein the catalyst used in the step i) is anhydrous sodium acetate, preferably 0.05 wt % of anhydrous sodium acetate, and wherein the coupling agent used to coupling reaction in step vi) is selected from N,N′-dicyclohexyl carbodiimide (DCC), 1,3-di-p-tolyl carbodiimide or 1-ethyl-3-(3-dimethyl aminopropyl) carbodiimide (EDC or EDC·HCl), more preferably EDC, and the catalyst optionally used in step vi) is selected from N-hydroxysuccinimide, N-hydroxy benzotriazole, 4-dimethyl aminopyridine (DMAP) with carbodiimide derivatives, more preferably without catalyst in case of EDC.

17. The process according to point 15 or 16, wherein the aqueous concentration of the succinated PVA solution is kept below 3% by weight during the synthesis.

18. The mucoadhesive polymeric prodrug according to points 1 to 6 or the self-assembled nanoparticles of mucoadhesive polymeric prodrug according to points 7 to 14 obtainable by the process according to point 15.

19. The mucoadhesive polymeric prodrug according to points 1 to 6 or the self-assembled nanoparticles of mucoadhesive polymeric prodrug according to points 7 to 14 for use in the treatment of bladder, breast, lung, stomach and ovarian cancer.

### BRIEF DESCRIPTION OF THE DRAWINGS

FIG. 1 shows the scheme of the succinylation reaction of PVA and conjugation reaction of DOX (Compound A) and cysteamine (Compound B) (through amide bonds) with PVA-SA in addition to the self-assembling formation of nanoparticles by crosslinking of OH group and COOH group by EDC (through ester bonds). Said scheme is to be construed as an embodiment of the invention.

FIG. 2 shows the Debye plot to measure the molecular weight of the initial PVA.

FIG. 3 shows the substitution degree of PVA-SA samples as a function of molar ratio (%) of reacted succinic anhydride (SA) with OH of PVA.

FIG. 4 shows the FTIR spectra of initial PVA and modified forms.

FIG. 5 shows the change in the viscosity as a function of increasing the concentration of PVA-SA solutions due to increasing crosslinking ratio (A), the change in the turbidity (black) and particle size (gray) values of PVA-SA with EDC as a function of time due to particle formation by ester bond crosslinking (B).

FIG. 6a shows the TEM of the prepared polymeric prodrug with the corresponding size distribution histogram.

FIG. 6b shows the effect of cross-link density on the particle size of the polymeric particles.

FIG. 7 shows the DSC curve of PVA, PVA-SA, and conjugated particles.

FIG. 8 shows curves of the PVA-based hydrogel samples with water desorption enthalpy (ΔH_{w}) values (grey columns).

FIG. 9 shows the water contact angle measurement of the prepared polymer films.

FIG. 10 shows the crosslinking of PVA-SA-CYS with Mucin by the formation of a disulfide bond.

FIG. 11 shows EDX of a) cross-linking PVA-SA and b) PVA-SA-CYS c) PVA-SA-CYS-DOX.

FIG. 12 shows storage modulus (G) and loss modulus (G) as a function of strain (%) for the PVA-Cysteamine, mucin, and mixture.

FIG. 13 shows the change in the turbidity of the aqueous polymer dispersion as a function of time due to the mucoadhesive properties.

FIG. 14a shows the in-vitro releasing percent curve of pure DOX and PVA-SA-CYS-DOX with the different cross-linking ratios as well as the DOX release from liner polymer.

FIG. 14b shows the in-vitro releasing concentration (mg/mL) curve of pure DOX and PVA-SA-CYS-DOX with the different cross-linking ratios as well as the DOX release from liner polymer.

FIG. 15a shows the excitation curves for the same concentration of DOX; free Dox (black solid line), conjugated particles PVA-DOX NPs (gray dash line), and linear macromolecules of conjugated PVA-DOX LMs (black dash line).

FIG. 15b shows a photo of the dispersion state of DOX in the case of free DOX, PVA-DOX NP, and PVA-DOX LM.

FIG. 16 shows the nontoxic effect of the prepared polymeric prodrug.

FIG. 17 shows the in-vitro releasing curve of bare KETO and KETO from PVA-SA-CYS-KETO particles.

FIG. 18 shows the in-vitro releasing curve of bare HBA and HBA from PVA-SA-CYS-HBA particles.

### DETAILED DESCRIPTION OF THE INVENTION

According to the present invention, in addition to the conjugation of the biologically active compounds, the succinyl group provides suitable crosslinking groups (COOH) with the free (unmodified) OH groups of PVA backbone by the formation of an ester bond via the use of EDC as a coupling agent. In this way the drug release kinetic can also be varied by the cross-link density of the obtained particles, furthermore, the formed polymeric particles can protect the encapsulated drug from the environmental condition. In other words, the conjugation reaction of the biologically active compounds [e.g. doxorubicin (DOX)] to succinated PVA will be accompanied by self-assembling formation of nanoparticles by crosslinking of OH with COOH groups of the partially succinated polyvinyl alcohol.

The present invention relates to nanoparticles of mucoadhesive polymeric prodrug comprising a partially succinated polyvinyl alcohol (PVA-SA) polymer wherein the simultaneous presence of OH and COOH functional groups on the polymer backbone and the proper aqueous concentration of the polymer enable the linear polymer macromolecules for the self-assembling formation of nanoparticles in the presence of a coupling agent.

The present invention relates to nanoparticles of mucoadhesive polymeric prodrug comprising a partially succinated polyvinyl alcohol (PVA-SA) and biologically active compounds having at least one group selected from amino, carboxyl, and hydroxyl groups, where the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds, and wherein one of the biologically active compounds is an aminothiol, and wherein the polymeric prodrug has crosslinking ester groups formed between the carboxyl and hydroxyl groups of the partially succinated polyvinyl alcohol.

The present invention also relates to a method for producing of said nanoparticles of the mucoadhesive polymeric prodrug, wherein the method comprises self-assembling formation of nanoparticles in combination with the coupling reaction of the biologically active compounds including anticancer, antifibrotic, antibacterial compounds, and aminothiol compound to the partially succinated polyvinyl alcohol, and where the particles with prolonged drug release properties can attach to mucous membranes via strong disulfide bonds.

The present invention also relates to mucoadhesive polymeric prodrug as antifibrotic, anticancer antibacterial and/or antibiotic drug advantageously can be used to treat certain types of bladder, breast, lung, stomach, and ovarian cancer.

Examples of said biologically active compounds include antifibrotic, anticancer, antibacterial, and antibiotic compounds, and aminothiol compound.

In an embodiment of the invention said biologically active compounds are cysteamine (CYS) as the aminothiol compound and one compound selected from the group consisting of doxorubicin (DOX), ketoprofen (KETO), or 4-hydroxybenzyl alcohol (HBA).

In an embodiment of the invention said biologically active compounds are cysteamine (CYS) as the aminothiol compound and doxorubicin (DOX).

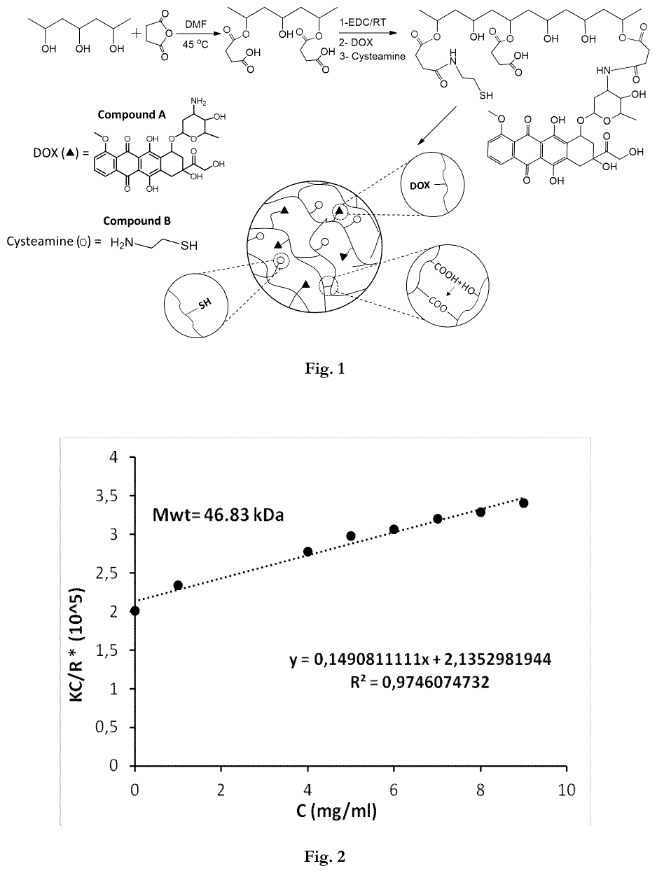

The chemical structure of the DOX drug (Compound A, as described in FIG. 1.) consists of aglyconic and sugar moieties. The aglycone includes a tetracyclic ring with adjacent quinone-hydroquinone groups, a methoxy substituent, and a short side chain with a carbonyl group and primary alcohol as terminate. The sugar, known as daunosamine, is bonded to one of the rings via a glycosidic link and consists of a 3-amino-2,3,6-trideoxy-L-fucosyl moiety [C. Carvalho et al., Curr. Med. Chem. 2009, 16, 3267-3285]. The structure of DOX can facilitate its interaction with DNA. Intercalation occurs as a result of the existence of anthraquinone rings, which allow the molecule to intercalate into the DNA [D. T. Breslin et al., Biochemistg 1997, 36, 10463-10473; S. J. Beckford and D. W. Dixon, J. Biomol. Struct. Dyn. 2012, 29, 1065-1080]. Moreover, the presence of daunosamine leads to the formation of a covalent aminal (N—C—N) bond in the DNA minor groove between the 3′NH₂ group and the N2 of the guanine base [A. H. Wang et al., Biochemistry 1991, 30, 3812-3815; S. M. Cutts et al., IUBMB life 2005, 57, 73-81; M. Ugarenko et al., Biochem. Pharmacol. 2010, 79, 339-349]. Also, the formation of a hydrogen bond between the hydroxyl group of the side chain and the corresponding DNA strand aids in the stabilization of the DOX-DNA mono-adduct formed [S. M. Cutts et al., IUBMB life 2005, 57, 73-81; M. Ugarenko et al., Biochem. Pharmacol. 2010, 79, 339-349].

DOX (Compound A) is one of the anthracycline family of antibiotics. Two proposed mechanisms for the anticancer effect of DOX (Compound A) have been confirmed. The first mechanism is DNA intercalation and the subsequent disruption of topoisomerase-II-mediated DNA repair for which potential pharmacogenes are MLH1, TOP2A, ERCC2, MSH2, and TP53 genes [K. M. Tewey et al., Science 1984, 226, 466-468]. The second mechanism includes the oxidative stress to cellular membranes, DNA, and proteins [M. Tokarska-Schlattner et al., J. Mol. Cell. Cardiol. 2006, 41, 389-405] for which potential genes involve nitric oxide synthases, NADH dehydrogenases, glutathione peroxidase, catalase, xanthine oxidase, and superoxide dismutase [J. Pawlowska et al., Oncol. Res. 2003, 13, 245-252].

The polyvinyl alcohol (PVA) polymer that is used in the present invention as an initial polymer for polymeric prodrug conjugate, consists primarily of 1,3-diol linkage, but depending on the polymerization conditions of the vinyl ester precursor, a few percent of 1,2-diols exist.

In an embodiment, PVA is used in the present invention for polymeric prodrug conjugate is represented by the following general formula:

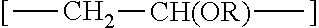

where R can be independently selected at each occurrence from hydrogen, acetate, succinate, aminobutyrate, p-nitrophenyl carbonate, p-formyl phenyl carbonate, or a biologically active moiety, the molecular weight of initial PVA with 86-89% hydrolysis was determined at around 46 kDa. The chemical modification of the PVA backbone can be performed via the secondary alcoholic OH group that can be easily modified due to its nucleophilicity properties.

In an embodiment of the invention, R can be succinate moiety with free carboxylic terminal groups that can conjugate with a biologically active compound such as doxorubicin through the formation of an amide bond and succinate moiety here as spacer or linker between the OH group of PVA backbone and NH group of doxorubicin (FIG. 1). The formation of amide bond provides the stability of doxorubicin inside the body due to the hydrolysis of amide bond is not an easy process, in addition to the reduce the side effect by the formation of this type of prodrug that provides the good carrier and protected candidate for doxorubicin. Reduce of the side effect of the drug in the present invention depends mainly in the following factor: slow-releasing of biologically active compound from the polymeric prodrug that enhances the prolongation of therapeutic effect and avoids frequently repeated-dose administration, mucoadhesive properties that enhances the residence time and avoid rapid elimination, the nanosized particles of the polymeric prodrug that enhances the surface area of drug and prevents accumulation, inactive form (prodrug) suitable for any type of administration routes to avoid the damage of tissue during the transportation and the hydrophobization that enhanced permeability and retention (EPR) effect.

In an embodiment of the invention, the PVA content used for polymeric prodrug conjugate may depend on the applied substitution reaction conditions.

The drug releasing from the nanoparticles of the invention can be adjusted by variation of crosslinking density or carboxylic group content.

As it mentioned above, the present invention also related to the method for producing of said nanoparticles of the mucoadhesive polymeric prodrug, said method comprises the following steps:
(a) dispersing the 10-20 μm PVA particles in an organic solvent and then adding the calculated amount of succinic anhydride (between 3.4 to 53.8 molar % to hydroxyl content) and 0.05 wt % of anhydrous sod. acetate as a catalyst; here we have to describe that this way the modification only occurs on the surface of undissolved particles and not the whole amount of available OH groups convert into COOH groups. Therefore, the OH/COOH ratio and thus the cross-link density can be adjusted;
(b) stirring the reaction mixture obtained in step (a) for 24 hours at 45° C.;
(c) removing the unreacted compounds and quenching the reaction of the solution obtained in step (b) and decantation the solvent to get the modified polymer particles, that is the particles of the partially succinated polyvinyl alcohol;
(d) purifying the modified polymer obtained in step (c) and drying under the vacuum.
(e) dissolving the modified polymer obtained in step (d) and biologically active compounds, which are an aminothiol and a biologically active compound having at least one group selected from amino, carboxyl, and hydroxyl groups, in distilled water;
(f) adding the coupling agent (50 mol % excess referred to COOH content) (see later), optionally with a catalyst to the solution obtained in step (e);
(g) stirring the solution obtained in step (f) for 3 hours at room temperature;
(h) removing the unreacted biologically active compounds and the coupling agent from the solution obtained in step (g) to get the desired polymeric prodrug;
(i) purifying the obtained polymeric prodrug obtained in step (h) and drying under the vacuum.

In an embodiment, in step (a) the organic solvent can be dimethylformamide (DMF) or dimethyl sulfoxide (DMSO) but in the case of using DMSO, the temperature should be 65° C. and using triethylamine (TEA).

In another embodiment, to remove unreacted compound and quenching the reaction in step (b) is performed via adding an excess amount of diethyl ether to the reaction mixture that leads to precipitate of the modified polymer.

In yet another embodiment, the purification of modified polymer in step (d) is performed via redissolving of modified polymer in DMF and reprecipitating by diethyl ether two times followed by washing the precipitate with acetone and ethanol.

In an embodiment of the invention, the aminothiol is cysteamine, and the biologically active compound having at least one group selected from amino, carboxyl, and hydroxyl groups is selected from the group consisting of doxorubicin (DOX), ketoprofen (KETO), or 4-hydroxybenzyl alcohol (HBA).

In an embodiment, the biologically active compound having at least one group selected from amino, carboxyl, and hydroxyl groups is doxorubicin (DOX). The concentration of the released DOX is varied between 0.011 and 0.0044 mg/mL depending on the cross-link density (1-9%) of the partially succinated polyvinyl alcohol.

In an embodiment of the present invention, the coupling agent that can be used in the coupling reaction in step (f) can be selected from N,N′-dicyclohexyl carbodiimide (DCC), 1,3-di-p-tolyl carbodiimide or 1-ethyl-3-(3-dimethyl aminopropyl) carbodiimide (EDC or EDC·HCl), in addition to using the catalyst such as N-hydroxysuccinimide, N-hydroxy benzotriazole, or 4-dimethyl aminopyridine (DMAP) with carbodiimide derivatives.

In another embodiment of the present invention, 1-ethyl-3-(3-dimethyl aminopropyl) carbodiimide (EDC) is a water-soluble coupling agent so the distilled water was used in step (e), however, an organic solvent such as DMF and DMSO can be used in case of using another coupling agent.

In an embodiment of the invention, the substitution (succination) degree of the partially substituted PVA is below 10 mol %. This ratio allows the formation of ester bonds between the OH and COOH groups of the partially substituted PVA. The cross-link density of the particles can be varied by the substitution degree.

In an embodiment of the invention, the aqueous concentration of the succinated PVA solution is below 3 wt. % during the synthesis in order to get separate nanoparticles containing dispersion having a viscosity of 0.8-2 mPas instead of the formation of viscous (>2 mPas) coherent gel structure.

In yet another embodiment of the present invention, the process of removing unreacted biologically active compounds, and the coupling agent in step (h) is performed via the addition of the excess amount of acetone or ethanol under strong magnetic stirring followed by centrifugation of the dispersed solution (15 min; 15000 rpm; Hermle centrifuge instrument), then the purification of the desired polymeric prodrug obtained in step (i) is performed via washing the precipitate two times with ethanol and one time with acetone.

In a particular embodiment, Compound A and Compound B constitute around 3.3% and 6.2% (wt. %) of the formulation (referred to the total amount of the polymeric prodrug) when the carboxylic content is around 9%, respectively.

In an embodiment of the invention, the size of the nanoparticles of the mucoadhesive polymeric prodrug is at most 260 nm which formed after 120 min reaction time, if the substitution degree is below 10% and the partially succinated PVA concentration is lower than 3 wt. %.

The above-mentioned self-assembling formation of nanoparticles ensures improved water dispersibility and prolonged release of conjugated/encapsulated drugs.

In an embodiment of the invention, the size of the nanoparticles of the mucoadhesive polymeric prodrug is 50 nm to 260 nm, and the viscosity of the nanodispersion containing said nanoparticles is less than 2 mPas. This size and viscosity enable the injectable applications of nanodispersion in medicine.

### EXAMPLES

### Example 1

### Preparation of the Partially Succinated PVA

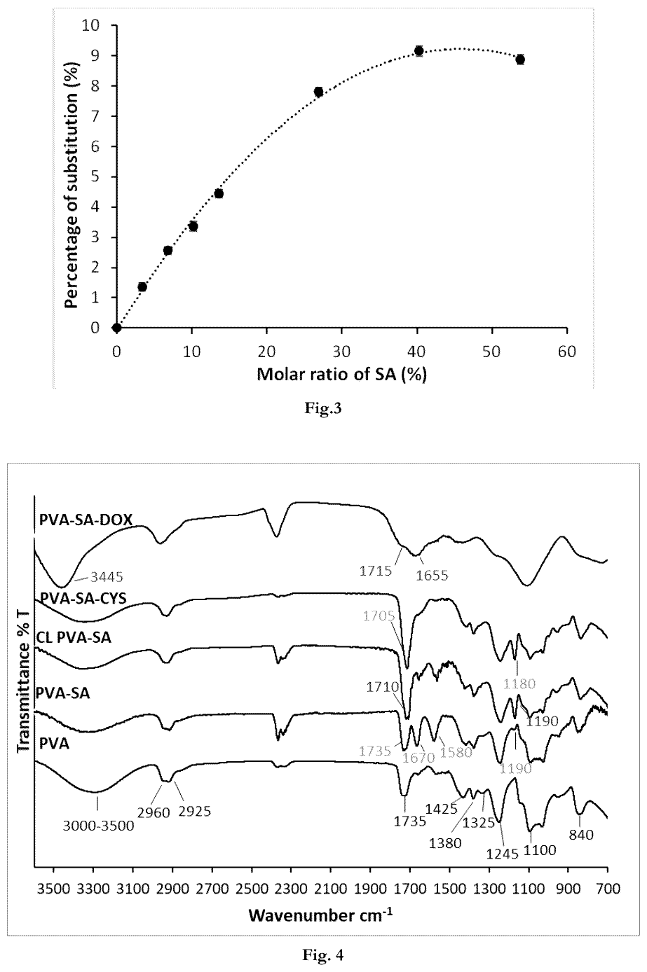

Functionalization of PVA with succinic anhydride (SA) as a source for carboxyl group as shown in FIG. 1 was applied with the different molar ratio of succinic anhydride according to the initial OH group of PVA that was calculated by using acetic anhydride/pyridine titration [T. Takács et al., Eur. Polym. J. 2020, 139, 109995]. 1 g of PVA with the different molar ratio (3.4, 6.8, 10.2, 13.6, 26.9, 40.3, and 53.8 molar %) of succinic anhydride for OH content of PVA was added to 8.6 ml of DMF followed by the addition of 0.05 g of anhydrous sodium acetate as a catalyst, the reaction mixture was left under continuous magnetic stirring at 45° C. for a duration of up to 24 h. The reaction product was then precipitated in an excess amount of diethyl ether. The obtained product was subsequently purified by repeated dissolution in DMF and precipitation in an excess amount of ether several times then washed with ether and then dried under vacuum until reaching for the constant weight. The initial molecular weight of PVA was determined by using DLS and according to Debye-plot the determined M_{w} is 46.83 kDa (FIG. 2). FIG. 3 shows the substitution degree as a function of increasing the molar ratio of SA, the carboxyl contain was measured by using acid-base titration that determined the mole number of the carboxyl group and by dividing this value by the mole number of the initial hydroxyl group of PVA, the percent of substitution can be calculated. The percentage of substitution increases with increasing the molar ratio of SA until reaches the maximum (saturation) value at around 9% with 40.3 molar % of SA. Due to the mild reaction condition according to the present invention, the percent of substitution was low because the reaction only occurred on the surface of undissolved PVA particles, however, use extreme conditions for substitution reaction will lead to the crosslinking of PVA by SA which was not preferred for our work as was studied by Zhou et al. [J. Appl. Polym. Sci. 2007, 103, 848-852].

### Example 2

### Preparation a Polymeric Prodrug Containing Doxorubicin as a Biologically Active Compound

The attachment of the carboxyl group to the PVA backbone provided a suitable candidate polymer for conjugation reaction by using EDC as a coupling agent, PVA-SA was reacted with cysteamine and DOX via amide bond formation in addition to the self-assembling formation of nanoparticles by ester bond between the carboxyl group and hydroxyl group (FIG. 1) as was reported by M. Tsakos et al. [Nat. Prod. Rep. 2015, 32, 605-632]. 100 mg of PVA-SA with different carboxylic content (0.5, 1.65, and 3.4 mmol/g of COOH group; which respects to 1.3, 4.4, and 9.1% substitution degree, respectively, as shown in FIG. 3) was dissolved in 10 ml of distilled water then 1.5 equivalent of EDC to COOH content (i.e. 14.4, 47.4, and 97.8 mg; which respects to 0.075, 0.248, and 0.51 mmol, respectively), 2 mg of DOX, and 1 equivalent of cysteamine to COOH content (3.9, 7.3, and 9.6 mg; which respects to 0.05, 0.165, and 0.34 mmol, respectively) were added to the polymer solution and then left under magnetic stirring (300 rpm) for 3 h at room temperature. After the reaction time, the reaction mixture was added to an excess amount of ethanol to precipitate the conjugated product followed by centrifugation (10000 rpm×10 min) to remove the solvent and washed by the mixture of acetone and water with ratio (3:1), the precipitate was left to dry under the vacuum, the absorbance of the supernatant was measured by UV-spectroscopy to calculate unbound DOX and the percent of binding (ranged from 80% to 90%) according to (Eq. 1).

$$Percent of binding ( \% ) = \frac{Total DOX - unbound DOX}{Total DOX} \times 100 \qquad ( Eq . 1 )$$

The successful reactions were confirmed by using FTIR measurements as presented in FIG. 4.

FIG. 4 shows the FTIR spectra of PVA, PVA-SA, and conjugated products with cysteamine and DOX. The main characteristic peaks of PVA are 3280 (O—H stretching vibration), 2960 (CH₂ asymmetric stretching vibration), 2925 (CH₂ symmetric stretching vibration) 1735 (C═O carbonyl stretch), 1425 (C—H bending vibration of CH₂), 1380 (C—H deformation vibration) 1325 (CH₂ wagging vibration), 1245 (C—O—C stretching vibration), 1100 (C—O stretching of acetyl groups), and 840 cm⁻¹ (C—C stretching vibration) [N. V. Bhat et al., Nucl. Instrum. Methods Phys. Res. B 2005, 237, 585-592; A. Kharazmi et al., Beilstein J. Nanotechnol. 2015, 6, 529-536].

When the OH groups of the PVA backbone are substituted by succinyl groups, the OH stretch band of pure PVA (˜3280 cm⁻¹) diminishes, the variation between these two spectra is characterized by the appearance of significant absorption bands at 1735 and 1670 cm⁻¹, which are related to the esteric carbonyls and carboxylate moieties, respectively [L. Stipniece et al., J. Cryst. Growth. 2016, 444, 14-20]. The new peak at 1580 cm-1 was due to the asymmetric stretching vibration of carboxylate RCOO⁻ [B. Zhang et al., Carbohydr. Polym. 2011, 84, 1276-1281]. The new absorption band between 1150-1190 cm⁻¹ for the PVA-SA arises due to C—O stretching vibrations of the ester group [H.-M. Guan et al., J. Membr. Si. 2006, 268, 113-122]. This evidences that the successful insertion of succinic moiety into the PVA side chains has occurred.

The conjugation reaction of PVA-SA with cysteamine was confirmed by the disappearance of the band at 1670 and 1580 cm⁻¹ that related to carboxylate moiety and asymmetric stretching vibration of carboxylate RCOO⁻, besides, shift and increase the intensity of the carbonyl band (C═O) at 1710 cm⁻¹ due to the formation of an amide bond between the carboxylate group of PVA-SA and NH₂ group of cysteamine. And also, the appearance of a new peck at 1180 cm⁻¹ was due to C—N stretching vibration which confirms the successful occurring of the conjugation reaction.

The successful conjugation reaction between PVA-SA and DOX was confirmed by the appearance of the amide band (C═O) at 1655 cm⁻¹ and ester band (C═O) at 1715 cm⁻¹ means the occurrence of the conjugation reaction and self-assembling formation of nanoparticles, respectively. The broad peak at around 3445 cm⁻¹ results from the OH stretching vibration of DOX moiety that confirms the conjugation reaction occurred via amide bond formation between NH₂ of DOX with COOH of PVA-SA (A. Vigevani, M. J. Williamson, Analytical Profiles of Drug Substances, 1981, 9, 245-274).

Self-assembling formation of nanoparticles by crosslinking of pendant OH and COOH groups was confirmed by decreasing of the band that related to carboxylate moiety of succinate and the appearance of a new absorption band between 1150-1190 cm⁻¹ due to C—O stretching vibrations of the ester group that confirmed the crosslinking by ester bond [M. Tsakos et al., Nat. Prod. Rep. 2015, 32, 605-632].

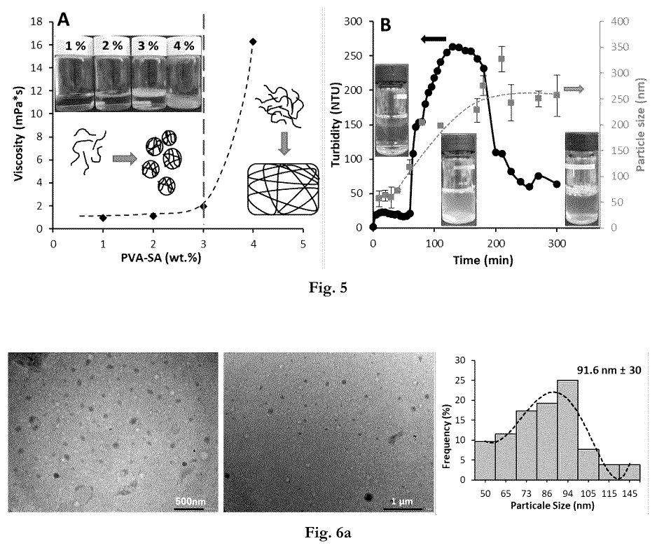

To prepare self-assembled nanoparticles, the change of viscosity of PVA-SA solutions was studied to determine the maximum polymer concentration that will be suitable to form separate particles instead of the formation of crosslinking bulk phase. For this examination, different PVA-SA solutions were prepared followed by adding the proper amount of EDC. FIG. 5A shows the change in the viscosity as a function of polymer solution concentration, the turbid solution was formed in the case of 1, 2, and 3% PVA-SA solutions with low viscosity values but after that, the viscosity was suddenly increased to 16.3 mPa-s indicating the formation of crosslinking hydrogel instead of the formation of separate polymeric particles as shown in 4% PVA-SA solution. These results show that the critical polymer concentration of our modified polymer is around 3% in water, below this concentration separate polymeric particles can be obtained, while at higher concentration values the macromolecule chains provide a cross-linked hydrogel sample.

The above-presented self-assembling formation of nanoparticles was also confirmed by turbidity measurement, 2 wt % of PVA-SA was prepared in an aqueous solution and after adding EDC as a reagent, the turbidity was monitored with the time. FIG. 5B shows a sudden increase in the turbidity was observed after adding EDC (˜20 NTU) because activation of COOH groups began with EDC and the turbidity showed a constant value before a significant increase after 60 min of measurement. After 130 min, the turbidity reached a plateau value and become constant again with increasing time until 180 min after that decrease again due to the coagulation of the particles. DLS measurement was performed to check the changes in the particle size of the prepared nanoparticles with the time and the corresponding data was also presented with the grey color in FIG. 5B. The particle size shows the tendency to increase with time, and the maximum particle size was −200 nm during the high turbidity value (between 120 to 170 min) while the maximum particle size was 256 nm after 300 min.

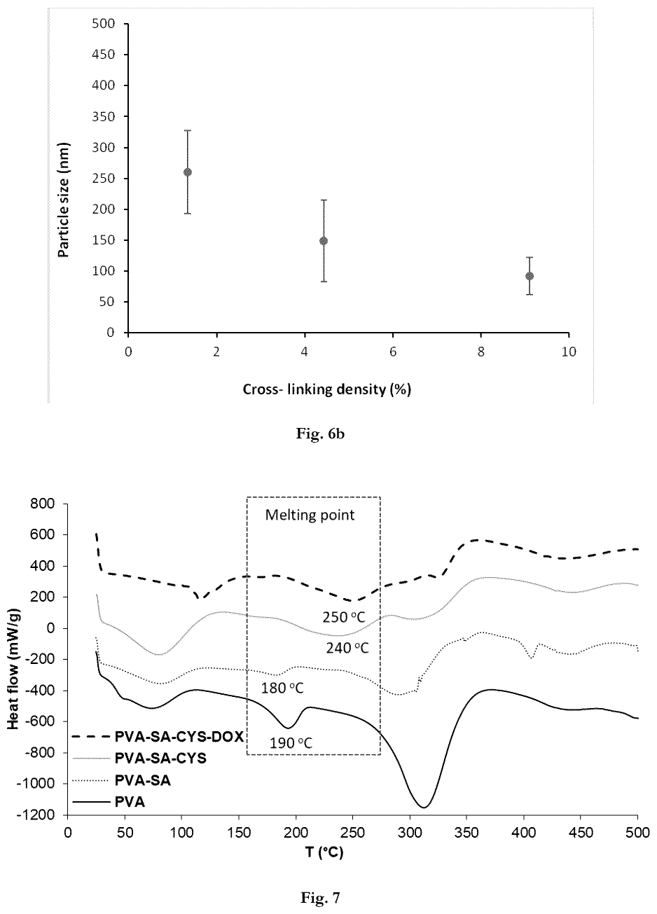

FIG. 6a shows a representative TEM image of spherical polymeric prodrug particles at different magnification and particle size distribution histogram with the mean particle size at around 92 nm. The particle size of the prepared polymeric spheres can be varied by the crosslink density: the increased COOH content (from 1 to 9%) caused a decrease in the measured particle size (from ˜260 to ˜90 nm) due to the formation of more ester bonds in the particles (FIG. 6b).

The thermal behaviour of the initial PVA and its modified derivatives were investigated with differential scanning calorimetry (DSC) measurements. The dried form of the polymer samples was used for the thermo-analytical measurements. The samples were heated from 25 to 500° C. at a heating rate of 5° C./min using a Mettler-Toledo 822e differential scanning calorimeter. FIG. 7 shows the difference in DSC curves of initial polymer and conjugated particles, the melting point of conjugated particles is higher compared to the initial one due to the formation of crosslinking particles that enhance the thermal properties of prepared particles in addition to increase the hydrophobicity that was confirmed by water contact angle measurement on the prepared films as presented in FIG. 9. The conjugated particles (PVA-SA-CYS) show a high contact angle at around 910 but in the case of initial and modified PVA were 550 and 57°, respectively.

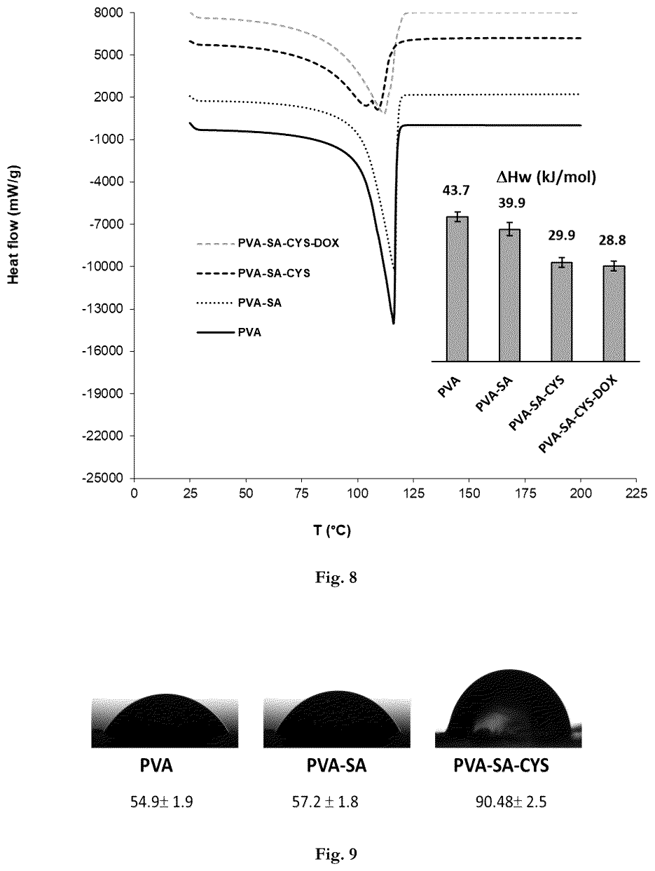

To prove the change in the hydrophobization besides the contact angle measurement and further recognized the changes in the thermal properties, the water heat evaporation and the desorption enthalpy value for the water content were measured by DSC measurements. The samples were swelled in dist. H₂O to prepare 2 wt % hydrogels and left overnight to reach the dissolution/swelling equilibrium. Then, the prepared solutions were centrifuged using (Eppendorf miniSpin Instrument) for 10 min with a speed of 14,500 rpm; the supernatant was removed and the obtained sediment was measured. The measurements were performed in the temperature range of 25-200° C. with a heating rate of 5° C./min. FIG. 8 shows the water evaporation curves and corresponding desorption enthalpy values (grey columns). Endothermic peaks were observed in all cases after the water content evaporation of the gels. For the evaluation of DSC curves, the water vaporization enthalpy (ΔH_{w}) values were calculated from the integration of the areas under the peaks, in case of conjugated particles show low desorption value at around 29 kJ/mol compared to the initial polymer and modified one with value at around 43.7 and 39.9 kJ/mol, respectively. To conclude the obtained results from thermal measurements, the coupling reaction of anticancer agent and aminothiol in addition to crosslinking increases the hydrophobicity of prepared polymeric prodrug that led to enhance thermal properties.

### Example 3

### Testing the Mucoadhesive Properties of the Prepared Polymeric Prodrug

The mucoadhesive properties of the formed particles were ensured by the terminal thiol groups (FIG. 1.). The thiol content of the thiolate sample was determined by Volhard's silver nitrate method. 100 mg of thiolate PVA (PVA-SA-CYS) was added to 25 ml of 0.01 M silver nitrate solution. The reaction mixture was covered well to avoid the light and stirred for 4 h before titration. After that time, unreacted silver nitrate was determined by titration with standardized 0.01 M KSCN using 0.2 g of ferric nitrate as an indicator. The endpoint of the titration was estimated when forming of the red colour complex [Fe(SCN)₆³⁻] from an excess of thiocyanate anion (SCN) and the ferric ion (Fe³⁺) of the indicator [B. Gupta et al., J. Appl. Polym. Sd. 2013, 129, 815-821]. The amount of thiol content was calculated from the amount of unreacted silver ion as shown in (Eq. 2).

$$Thiol content = \frac{0.25 - ( C * V )}{wt of PVA} mmol / g \qquad ( Eq . 2 )$$

This measurement depends on the amount of unreacted silver ion that can be determined by titration with standardized 0.01 M KSCN. Thiol content in the case of PVA-SA-CYS (9%) was 0.8 mmol/g compared to PVA-SA-CYS (4%) and PVA-SA-CYS (1%) were 0.2 and 0.075 mmol/g, respectively. The conjugation with PVA-SA (9% carboxyl content) increases the mucoadhesive properties of the prepared particle by increasing the thiol group that plays a key role in the adhesion of the conjugated particle to the membrane as will be presented in the mucoadhesive section. In addition to increasing the crosslinking density by increase the chance of reaction between the OH group of PVA and COOH group of substituted SA that leads to reduce the particle size of the prepared particles.

EDX measurements were used to detect the presence of the elements in the different conjugated forms, PVA-SA-CYS particles show the appearance of the sulfur and nitrogen element that confirms the conjugation of cysteamine to PVA-SA by coupling reaction with EDC. In the case of PVA-SA-CYS-DOX, the sulfur and nitrogen elements also appeared due to Cysteamine and DOX that contain nitrogen in addition to carbon as shown in FIG. 11.

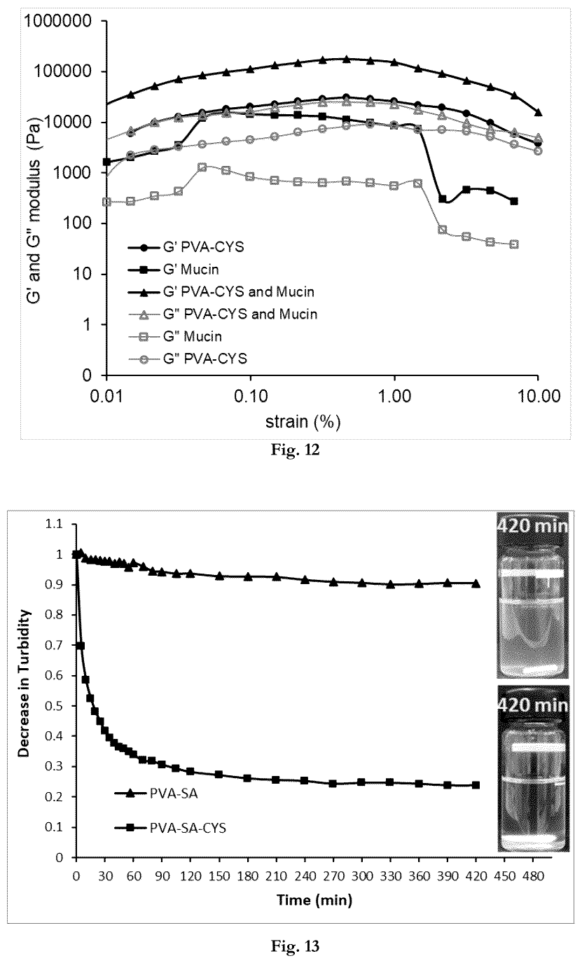

The mucoadhesive properties of the thiolate polymer were also evidenced via oscillatory rheology measurements using a Physica MCR 301 rheometer (Anton Paar, Graz, Austria). The measurement system was of cone and plate type (cone angle 0.99°, gap height in the middle of the cone 0.054 mm, and diameter 11.95 mm). In our measurement, 1% w/v of PVA-SA-CYS and mucin were prepared by using dist. H₂O, the mixture of PVA-SA-CYS and mucin was prepared by mixing of previously prepared solutions with an identical amount in addition to the final concentration in the mixture was 1% w/v and the gelation was followed after overnight. The gelation of the PVA-SA-CYS, mucin, and mixture was followed at an angular frequency of 10 Hz at 25° C., and storage modulus (G) and loss modulus (G″) were determined over the strain range from 0.1 to 10%. The storage modulus (G′) provides information about the elastic nature of the polymer while the loss modulus (G″) represents the viscous nature of the polymer. FIG. 12 shown the storage modulus of PVA-CYS-Mucin varied from 10⁴ to 10⁵ Pa but the storage moduli of PVA-CYS and Mucin varied from 10³ to 10⁴ Pa while the corresponding data were presented in Table 1.

TABLE 1
Data of storage modulus (G′), loss modulus (G″), and
complex modulus (G*) of PVA-SA-CYS (1%) and mucin (1%)

| Sample | G′ (kPa) | G″(kPa) | G*(kPa) |
|---|---|---|---|
| 1% PVA-CYS | 24.7 | 7.55 | 32.25 |
| 1% Mucin | 12.4 | 0.78 | 13.18 |
| 1% Mucin and PVA-CYS | 158.3 | 23.2 | 181.5 |

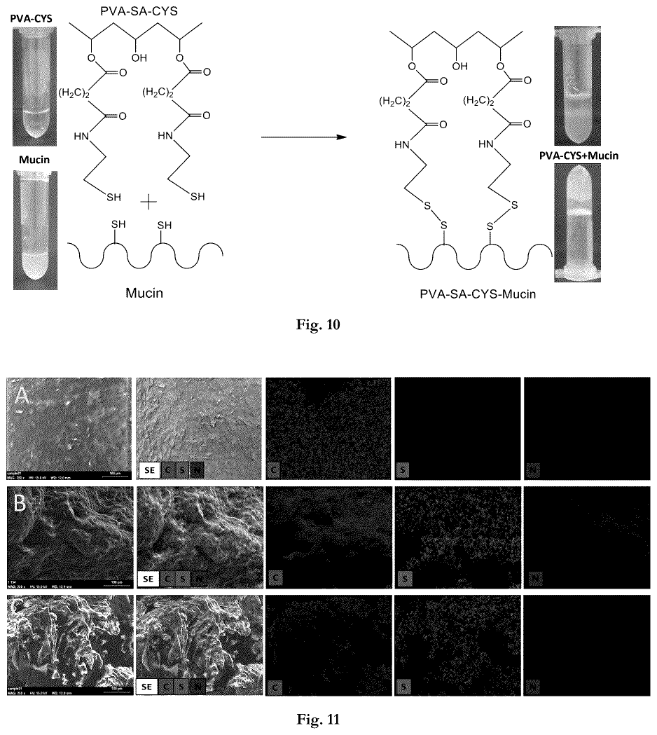

The ‘sum’ of loss and storage modulus is the so-called complex modulus (G*). The PVA-CYS-Mucin suspension had much higher G′ values than PVA-CYS and Mucin, indicating the formation of a more entangled network by disulfide cross-linking bond that leads to the formation of the gel as presented in FIG. 10. With the same approach, the loss modulus of PVA-CYS-Mucin had higher values than PVA-CYS and Mucin. It can be concluded that the formation of the disulfide bond between the mucoadhesive polymer and Mucin increases the elasticity and viscosity that approve the formation of this bond that will play a key role in the adhesion of conjugated particles of the desired site for releasing of DOX as shown in FIG. 13.

The formation of the disulfide bonds between our mucoadhesive polymer particles and the mucin-rich surface was also proved by surface adsorption measurements. During this experiment, a pig intestinal obtained from a local slaughterhouse as model mucus surface was immersed in 1 wt % of an agitated aqueous dispersion of mucoadhesive particles, and the decreasing turbidity was measured as a function of time. The same measurement was repeated with particles without cysteamine as control. Due to the continuous surface adsorption of particles on the intestinal membrane. FIG. 13 shows the in vitro mucoadhesive measurement by using the pig intestinal as tested membrane and the conjugated form of PVA with CYS and without CYS. The presence of cysteamine (CYS) lead to an increase of mucoadhesive properties of initial PVA and the turbidity was a dramatic decrease with time due to the adhesion of dispersed PVA-SA-CYS particles to the membrane by disulfide bond formation until reach full adsorption with the maximum decreasing 76.2% compared to initial PVA-SA particles with maximum decreasing 9.5%. In the case of PVA-SA-CYS, around 60% of particles were adsorbed on the intestinal surface after 1 h of measurement that means the existence of thiol groups of cysteamine moiety permits the formation of covalent bonds with cysteine-rich subdomains of the mucus layer, resulting in increased residence time of conjugated particles and improved bioavailability.

### Example 4

Testing the Doxorubicin Release from the Prepared Polymeric Prodrug

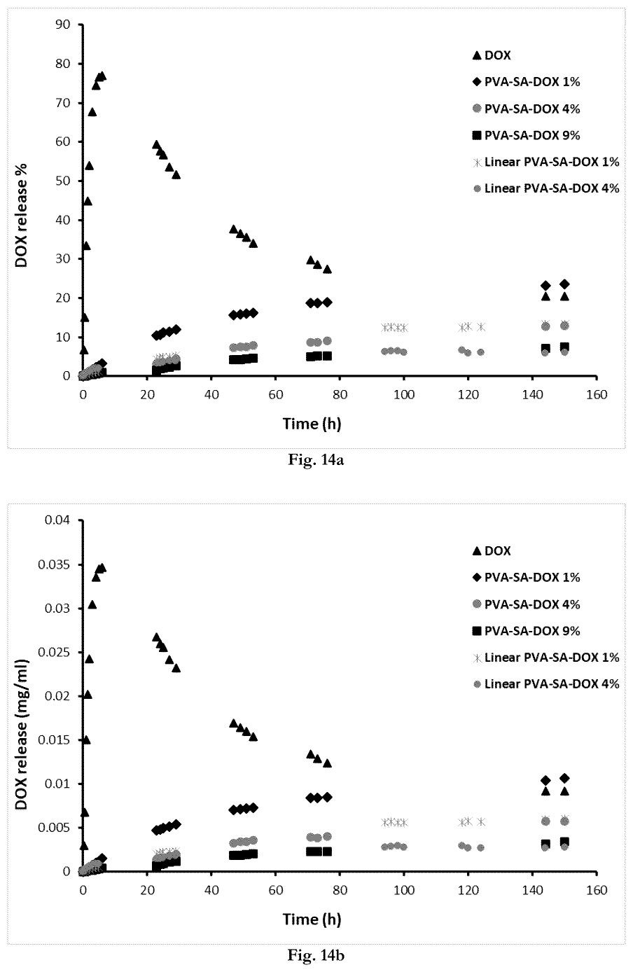

The DOX release was measured according to the following: the in vitro experiments of the DOX releasing were performed using a cellulose membrane (Sigma-Aldrich, avg. flat width 10 mm (0.4 in.)). The released DOX amount from the bare and conjugated form was spectrophotometrically measured by monitoring the change in the intensity of the characteristic absorbance peak of DOX at λ=481 nm. DOX concentration is directly proportional to the maximal absorption values estimated at 481 nm [C_{DOX} (mg/ml) (A at 481 nm)/18.836; R²=0.996], according to the calibration curve previously determined within the range of 0-0.066 mg/ml. Solid powder of pure drug and conjugated form (corresponding to 0.045 mg/ml of DOX) were inserted in a cellulose membrane that was closed and dropped into 20 ml of a PBS buffer solution with pH=7.4 (37° C.; 0.9 wt % NaCl content). 3 ml of the aqueous solution was pulled from the release media at selected interval times and DOX concentration was measured. The release profiles of DOX released from different forms are shown in FIG. 14a in percent form and FIG. 14b in concentration form, releasing from the bare DOX was too fast a process with a maximum releasing percent of 78%. After one day of DOX release, the concentration of DOX in PBS buffer solution at 37° C. was continuously decreased due to DOX decomposition, which was completely consistent with the reported data (M. J. H. Janssen et al., Int. J. Pharm. 1985, 23, 1-11). According to Table 2, releasing from the bare DOX data were fitted with the Higuchi model that describes the drug release a diffusion process based on Fick's law which is square root time-dependent, assumes the diffusivity of the drug is constant.

TABLE 2
Interpretation of the release experiments using different kinetic models.

| | Zero-order | | First-order | | | | Hixson- | | Korsmeyer-Peppas | | |
|---|---|---|---|---|---|---|---|---|---|---|---|
| | model | | model | | Higuchi model | | Crowell model | | model | | |
| Sample | r² | k | r² | k | r² | k | r² | k | n | r² | k |
| Pure DOX | 0.849 | 13.11 | 0.936 | 0.2689 | 0.957 | 36.808 | 0.5558 | 0.0063 | 0.763 | 0.9375 | 26.18 |
| PVA-SA-CYS- | 0.819 | 0.155 | 0.846 | 0.0018 | 0.9747 | 2.1528 | 0.5907 | 0.0134 | 0.793 | 0.9756 | 0.653 |
| DOX (1%) | | | | | | | | | | | |
| PVA-SA-CYS- | 0.931 | 0.091 | 0.941 | 0.0009 | 0.9771 | 1.1812 | 0.7105 | 0.0122 | 0.861 | 0.9788 | 0.209 |
| DOX (4%) | | | | | | | | | | | |
| PVA-SA-CYS- | 0.941 | 0.054 | 0.945 | 0.0005 | 0.9635 | 0.6955 | 0.6638 | 0.0104 | 0.768 | 0.9758 | 0.186 |
| DOX (9%) | | | | | | | | | | | |

In contrast, the release of DOX from conjugated form (PVA-SA-CYS-DOX) with different crosslinking densities (1, 4, and 9%, respectively) shows slow-releasing profiles due to the releasing process in these cases depending on the amide hydrolysis of DOX moiety from the conjugated form. Amide hydrolysis reactions are very much slower that is the reason for the releasing of DOX from conjugated forms are slow and taken a long period to achieve plateau value, the maximum DOX releasing after 7 days for PVA-SA-CYS-DOX (9% COOH), PVA-SA-CYS-DOX (4% COOH) and PVA-SA-CYS-DOX (1% COOH) is 9.8, 14.9 and 24.4%, respectively. According to Table 2, drug release data for all the conjugated forms of PVA-SA-CYS-DOX were fitted in the Korsmeyer-Peppas model. Korsmeyer and Peppas developed a basic relationship that explained the release of drugs from a polymer system that followed the form of dissolution and defined an equation as:

$${M}_{t} / {M}_{∞} = {Kt}^{n}$$

where Mₜ/M_{∞} is a fraction of drug released at time t, k is the release rate constant and n is the release exponent (or diffusional exponent). Based on an “n” value; the release exponent n<0.45 corresponds to a Fickian diffusion mechanism, whereas 0.45<n<0.89 corresponds to non-Fickian (anomalous) transport. Due to n<0.2 for a pure and physical mixture that means it is Fickian diffusion release, i.e. diffusion-controlled transport.

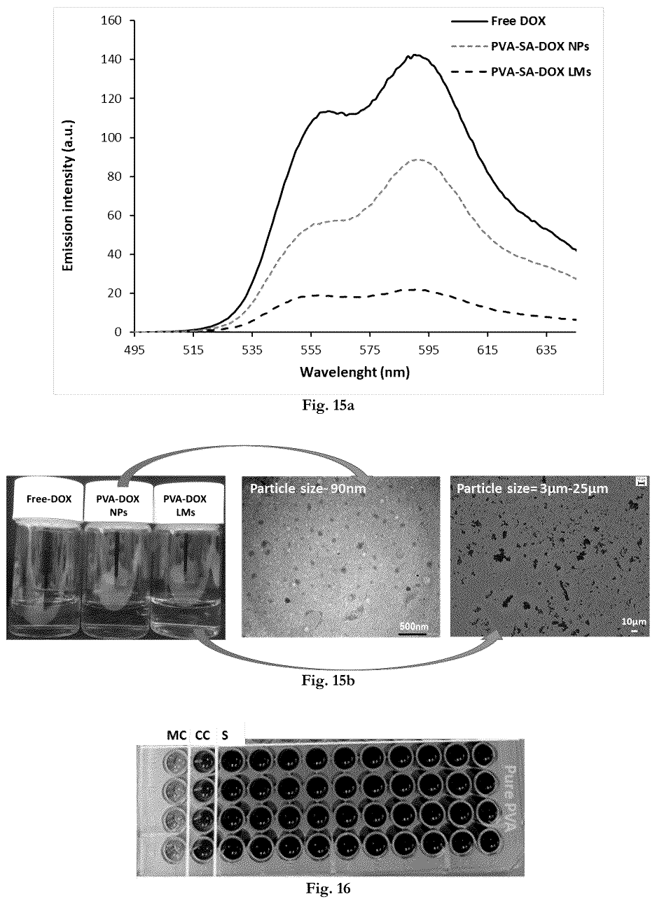

Based on an “n” value (diffusional exponent) between 0.45 and 0.89 corresponds to an anomalous diffusion (non-Fickian), or drug release mechanism is depending on both diffusion and erosion-controlled mechanisms [O. L. Freichel and B. C. Lippold, Eur. J. Pharm. Biopharm. 2000, 50, 345-351; C. K. Sackett and B. Narasimhan, Int. J. Pharm. 2011, 418, 104-114; A. C. Salome et al., Res. J. Pharm. Biol. Chem. Sd. 2013, 4, 97-103] that is mean the hydrolysis reaction of conjugated form in the first followed by diffusion of DOX drug. A reference measurement was also performed in order to prove the importance of the self-assembling formation of nanoparticles on the drug release properties. In this case, we synthesized linear macromolecules with conjugated DOX but without the formation of particles (linear PVA-SA-DOX 1 and 4%). In some previous papers and patents, DOX and other drugs were conjugated on the COOH containing polymers [K. M Camacho et al., Nanomedicine 2016, 11, 1139-1151; P. Jayaraman et al., Bull. Mater. Sd. 2016, 39, 201-207; K. M. Camacho et al., Polymer-Drug Conjugates for Combination Anticancer Therapy (WO2016145096A1), issued Sep. 15, 2016]. In this way, we represent the advantage of our solution compared to the “state of the art” report. If we compare the release profiles of DOX from self-assembled nanoparticles to the simply conjugated form (linear PVA-SA-DOX 1 and 4%), it can be seen that the DOX release is actually faster from the particles than from the conjugated form (linear PVA-SA-DOX 1 and 4%). This is due to the smaller particle size. The conjugation of DOX (Compound A) with modified polymer led to reducing its emission intensity and slight blue shift as shown in FIG. 15a. The three samples have the same concentration 0.2 mg in 5 ml of water and were sonicated for 10 min to be sure the complete dissolving and dispersing of DOX in an aqueous medium and excited at 481 nm but due to the conjugation of DOX in the case of PVA-SA-DOX NPs, the amount of free DOX and bonding state with polymer result in emission intensity and slight blue shift, also, in case of PVA-SA-DOX LMs, due to the large size and lack of dispersion in water led to the significant decrease of the emission intensity of DOX and blue shift compared to free DOX (reference sample) as shown in FIG. 15a and FIG. 15b. This is also clearly seen from the TEM and microscopic pictures in FIG. 15b. The DOX containing conjugated polymeric prodrug (PVA-SA-DOX LMs) forms irregular shapes 3-25 μm microparticles in the aqueous phase, while the sample with the self-assembling formation of nanoparticles behaviour provides nanoparticles with the mean particle size of 90 nm. This is very advantageous because previous studies have also shown that cellular internalization exhibited a strong dependence on particle size, smaller particles are better internalized by cancer cells compared to large particles with low cellular internalization [S. E. A. Gratton et al., Proc. Natl Acad. Sci. USA 2008, 105, 11613-11618]. Furthermore, the water stability of the nanoparticles is also better than the microparticles. Thus it can be concluded that our solution is suitable for the encapsulation and surface immobilization of different biologically active compounds with amino and/or carboxyl and/or hydroxyl groups and the particles are also able to prolong the drug release and dissolution.

The prepared polymeric prodrug has a non-toxic effect as shown in FIG. 16, in addition, prolong the time of releasing as shown in FIG. 14 which means it provides a safe administration way for DOX (Compound A) that has severe local tissue injury and necrosis in case of extravasation that requires wide excision of the affected area and skin grafting.

Finally, we also want to prove the applicability (conjugation and prolonged drug-release ability) of our solution in the case of carboxyl (Ketoprofen) and hydroxyl (4-hydroxybenzyl alcohol) group containing molecules.

### Example 5

### Preparation of a Polymeric Prodrug Containing Ketoprofen as a Biologically Active Compound

100 mg of PVA-SA (1% crosslinking ratio) was dissolved in 10 ml of distilled water, then 1 mg of ketoprofen (KETO, a drug with carboxylic acid group content) and 1 equivalent of cysteamine to COOH content (3.9 mg, 0.05 mmol) were added followed by adding of 1.5 equivalent of EDC to COOH content (14.4 mg, 0.075 mmol) and the reaction was left under continuous magnetic stirring for 3 h. The reaction mixture was added to the excess amount of ethanol or acetone and the precipitated product was collected by centrifugation and washing the product well with acetone, and then the product was dried under the vacuum.

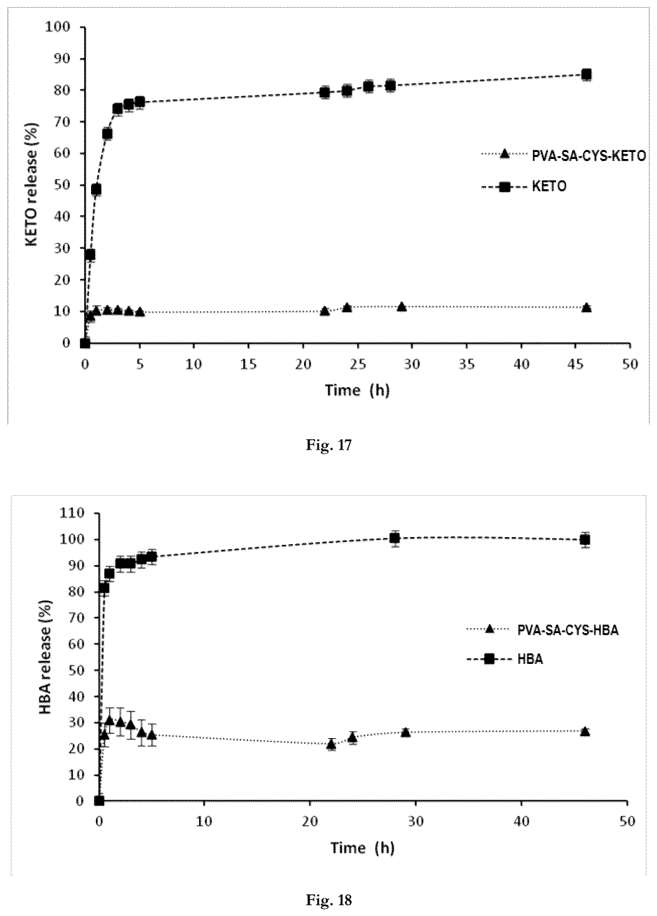

FIG. 17 shows the in-vitro drug-releasing profile of KETO, bare KETO (unconjugated form) shows the fast releasing process for the first 5 h of measurement, in contrast, the conjugated form of KETO shows the very slow releasing process form PVA-SA-CYS-KETO because of the releasing process is controlled by hydrolysis of drug moiety from the conjugated form followed by drug diffusion.

### Example 6

### Preparation of a Polymeric Prodrug Containing 4-Hydroxybenzyl Alcohol as a Biologically Active Compound

100 mg of PVA-SA (9% crosslinking ratio) was dissolved in 10 ml of distilled water, then 4 mg of 4-hydroxybenzyl alcohol (HBA, a drug with hydroxyl group content) and 1 equivalent of cysteamine to COOH content (9.6 mg, 0.34 mmol) were added followed by adding of 1.5 equivalent of EDC to COOH content (97.8 mg, 0.51 mmol) and the reaction was left under continuous magnetic stirring for 3 h. the reaction mixture was added to the excess amount of ethanol or acetone and the precipitated product was collected by centrifugation and washing the product well with acetone, and then the product was dried under the vacuum.

With the same strategy as in Example 5, the in-vitro drug-releasing profile of HBA was shown in FIG. 18. Bare HBA (unpaired form) shows a very rapid release process for the first 3 h of measurement due to the relatively high water solubility of HBA, however, the conjugated form of HBA shows the slow-releasing process from PVA-SA-CYS-HBA (conjugated form) due to the releasing process is controlled by hydrolysis of drug moiety from the conjugated form followed by drug diffusion.

It is also worth to note that besides the above presented antibacterial properties the antimicrobial behaviour also can be reached by the incorporation of silver and/or copper nanoparticles into the polymer matrix. Owing to their smaller size and large surface area, AgNPs have strong antimicrobial activity against different microorganisms like viruses, bacteria, and fungi [G. Franci et al., Molecules 2015, 20, 8856-8874]. AgNPs are also generally used as anti-inflammatory [A. Hebeish et al., Int. J. Biol. Macromol. 2014, 65, 509-515], anti-fungal [S. Medda et al., Appl. Nanosci. 2015, 5, 875-880], and anti-viral properties [A. Bekele et al., Foodborne Pathog. Dis. 2016, 13, 239-244]. Copper is another example that is really fascinating because of the simple access to metal, cheap, and one of the main trace elements in several living organisms. Antimicrobial copper kills 99.9% of many of these pathogens during 2 h of contact [M. Hans et al., Langmuir 2013, 29, 16160-16166], furthermore, in certain cases, it has better properties than other costly metals, such as silver and gold, which have antimicrobial activity [M. S. Usman et al., Int. J. Nanomedicine 2013, 8, 4467-44791. For example, the Cu NPs showed relatively higher antibacterial activity compared to the silver NPs against Bacillus subtilis (B. subtilis) and E. coli [K. Y. Yoon et al., Sci. Total Emiron. 2007, 373, 572-575; J. P. Ruparelia et al., Acta Biomater. 2008, 4, 707-716].

### INDUSTRIAL APPLICABILITY

Based on the foregoing it can be concluded that solution according to the present invention is suitable for the encapsulation and surface immobilization of different biologically active compounds with amino and/or carboxyl and/or hydroxyl groups and the particles are also able to prolong the drug release and dissolution.

1. A mucoadhesive polymeric prodrug comprising
a) a partially succinated polyvinyl alcohol (PVA-SA) polymer;
b) two or more biologically active compounds having at least one functional group selected from amino, carboxyl, and hydroxyl groups;
wherein one of the biologically active compounds is an aminothiol compound,
and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
and wherein the polymeric prodrug has crosslinking ester groups formed between the carboxyl and hydroxyl groups within the structure of the partially succinated polyvinyl alcohol.

2. The mucoadhesive polymeric prodrug as claimed in claim 1, wherein the molecular weight of the partially succinated PVA-SA polymer is between 20-80 kDa, more preferably 40-60 kDa.

3. The mucoadhesive polymeric prodrug as claimed in claim 1, wherein the substitution (succination) degree in the partially succinated PVA-SA polymer is below 10 mol %.

4. The mucoadhesive polymeric prodrug as claimed in claim 1, wherein the cross-link density is 1-9% in the partially succinated PVA-SA polymer.

5. The mucoadhesive polymeric prodrug as claimed in claim 1, wherein the biologically active compounds are cysteamine (CYS) as the aminothiol compound and one compound selected from the group consisting of doxorubicin (DOX), ketoprofen (KETO) and 4-hydroxybenzyl alcohol (HBA), more preferably DOX.

6. The mucoadhesive polymeric prodrug as claimed in claim 1, wherein DOX and cysteamine (CYS) constitute approx. 3.3% and 6.2% by weight of the formulation, respectively, referred to the total amount of the polymeric prodrug, when the carboxylic content is approx. 9% by weight.

7. Self-assembled nanoparticles of mucoadhesive polymeric prodrug comprising
a) a partially succinated PVA-SA polymer;
b) two or more biologically active compounds having at least one functional group selected from amino, carboxyl, and hydroxyl groups;
wherein one of the biologically active compounds is an aminothiol compound,
and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
and wherein the polymeric prodrug has crosslinking ester groups formed between the carboxyl and hydroxyl groups within the structure of the partially succinated polyvinyl alcohol.

8. The self-assembled nanoparticles of mucoadhesive polymeric prodrug as claimed in claim 7, wherein the size of the nanoparticles of the mucoadhesive polymeric prodrug is at most 260 nm, preferably 50 nm to 260 nm, the substitution degree is below 10% and the partially succinated PVA concentration is lower than 3% by weight.

9. The self-assembled nanoparticles of mucoadhesive polymeric prodrug as claimed in claim 7, wherein the nanoparticles have a size of 50-260 nm.

10. The self-assembled nanoparticles of mucoadhesive polymeric prodrug as claimed in claim 7, wherein the molecular weight of the partially succinated PVA-SA polymer is approx. 46 kDa.

11. The self-assembled nanoparticles of mucoadhesive polymeric prodrug as claimed in claim 7, wherein the substitution (succination) degree in the partially succinated PVA-SA polymer is below 10 mol %.

12. The self-assembled nanoparticles of mucoadhesive polymeric prodrug as claimed in claim 7, wherein the cross-link density is 1-9% in the partially succinated PVA-SA polymer.

13. The self-assembled nanoparticles of mucoadhesive polymeric prodrug as claimed in claim 7, wherein the biologically active compounds are CYS as the aminothiol compound and one compound selected from the group consisting of DOX, KETO and HBA, more preferably DOX.

14. The self-assembled nanoparticles of mucoadhesive polymeric prodrug as claimed in claim 7, wherein DOX and CYS constitute approx. 3.3% and 6.2% by weight of the formulation, respectively, referred to the total amount of the polymeric prodrug, when the carboxylic content is approx. 9% by weight.

15. A process for producing the mucoadhesive polymeric prodrug as claimed in claim 1, or the self-assembled nanoparticles of mucoadhesive polymeric prodrug comprising
a) a partially succinated PVA-SA polymer;
b) two or more biologically active compounds having at least one functional group selected from amino, carboxyl, and hydroxyl groups;
wherein one of the biologically active compounds is an aminothiol compound,
and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
and wherein the polymeric prodrug has crosslinking ester groups formed between the carboxyl and hydroxyl groups within the structure of the partially succinated polyvinyl alcohol, wherein the following steps are taken:
i) dispersing 10-20 μm PVA particles in an organic solvent and then adding calculated amount of succinic anhydride between 1 to 60 molar %, preferably 3 to 55 molar %, in particular e.g. 3.4 to 53.8 molar % to hydroxyl content, and a catalyst;
ii) optionally stirring the reaction mixture obtained in step i);
iii) optionally removing the unreacted compounds and quenching the reaction of the solution obtained in step ii) and decanting the solvent to obtain particles of the partially succinated PVA;
iv) optionally purifying the polymer obtained in step iii) and drying under the vacuum;
v) optionally dissolving the modified polymer obtained in step iv) and all of the biologically active compounds used in distilled water;
vi) adding a coupling agent, preferably in approx. 50 mol % excess referred to COOH content, optionally with a catalyst to the solution obtained in step v);
vii) optionally stirring the solution obtained in step vi);
viii) optionally removing the unreacted biologically active compounds and the coupling agent from the solution obtained in step vii) to obtain the desired polymeric prodrug;
ix) optionally purifying the obtained polymeric prodrug obtained in step viii) and drying under the vacuum.

16. The process as claimed in claim 15, wherein the catalyst used in the step i) is anhydrous sodium acetate, preferably 0.05 wt % of anhydrous sodium acetate, and wherein the coupling agent used to coupling reaction in step vi) is selected from N,N′-dicyclohexyl carbodiimide (DCC), 1,3-di-p-tolyl carbodiimide or 1-ethyl-3-(3-dimethyl aminopropyl) carbodiimide (EDC or EDC·HCl), more preferably EDC, and the catalyst optionally used in step vi) is selected from N-hydroxysuccinimide, N-hydroxy benzotriazole, 4-dimethyl aminopyridine (DMAP) with carbodiimide derivatives, more preferably without catalyst in case of EDC.

17. The process as claimed in claim 15, wherein the aqueous concentration of the succinated PVA solution is kept below 3% by weight during the synthesis.

18. (canceled)

19. A method of treating cancer comprising administering an effective amount of the mucoadhesive polymeric prodrug as claimed in claim 1 or the self-assembled nanoparticles of mucoadhesive polymeric prodrug comprising
a) a partially succinated PVA-SA polymer;
b) two or more biologically active compounds having at least one functional group selected from amino, carboxyl, and hydroxyl groups;
wherein one of the biologically active compounds is an aminothiol compound,
and wherein the biologically active compounds are linked to the partially succinated polyvinyl alcohol by ester or amide linkage formed between the hydroxyl or carboxyl groups of the partially succinated polyvinyl alcohol and the amino, carboxyl or hydroxyl groups of the biologically active compounds;
to a patient in need thereof.

20. The method according to claim 19, wherein said cancer is bladder, breast, lung, stomach and/or ovarian cancer.
